(19) United States
(12) Patent Application Publication
(10) Pub. No.: US 2024/0423896 A1
(43) Pub. Date: Dec. 26, 2024

(54) METHOD FOR IMPROVING THE TACTILE SENSATION OF DYED KERATINOUS FIBRES, IN PARTICULAR HUMAN HAIR

(71) Applicant: HENKEL AG & CO. KGAA, Düsseldorf (DE)

(72) Inventors: Constanze KRUCK, Grevenbroich (DE); Gabriele WESER, Essen (DE); Sandra HILBIG, Bochum (DE); Melanie MOCH, Dormagen (DE)

(21) Appl. No.: 18/689,892

(22) PCT Filed: Aug. 2, 2022
(86) PCT No.: PCT/EP2022/071657

§ 371 Date: Mar. 7, 2024

(30) Foreign Application Priority Data
Sep. 8, 2021 (DE) 10 2021 209 926.2

(51) Int. Cl. A61K 8/898; A61K 8/73; A61Q 5/00

(52) U.S. Cl. CPC A61K 8/898; A61K 8/732; A61Q 5/008; A61K 2800/43

(57) ABSTRACT

A method for improving the tactile sensation of keratinous fibers which have been dyed is described. At least one amino-functionalized silicone polymer and at least one pigment are applied to the keratinous fibers. A post-treatment agent is applied to the dyed keratinous fibers, where the post-treatment agent includes at least one sebum-adsorbing substance.

The present invention relates to a method for improving the tactile sensation of keratinous fibers which have been dyed by applying at least one amino-functionalized silicone polymer and at least one pigment, wherein a post-treatment agent is applied to the dyed keratinous fibers, which post-treatment agent contains at least one sebum-adsorbing substance.

This application relates secondly to a method for coloring and post-treating keratinous fibers, in particular human hair, in which first a coloring agent containing at least one aminosilicone and at least one pigment is used, and subsequently the post-treatment agent described above is applied.

The present application relates thirdly to a multicomponent packaging unit (kit-of-parts) which contains the previously described coloring agent and post-treatment agent in separately packaged containers.

The present application relates fourthly to the use of the above-described post-treatment agent for improving the tactile sensation of dyed keratin fibers.

Changing the shape and color of keratin material, in particular hair, represents an important area of modern cosmetics. To change the hair color, a person skilled in the art is familiar with a variety of dyeing systems depending on the dyeing requirements. Oxidation dyes are typically used for permanent, intense dyeing with good fastness properties and good gray coverage. Such coloring agents contain oxidation dye precursors, so-called developer components and coupler components, which together form the actual dyes under the influence of oxidizing agents, such as, for example, hydrogen peroxide. Oxidation dyes are characterized by very long-lasting color results.

When using direct dyes, dyes that are already fully formed diffuse from the dye into the hair fiber. In comparison with oxidative hair coloring, the colors obtained with direct dyes have a lower durability and a more rapid washing out. Dyeing with direct dyes usually remains on the hair for a period of between 5 and 20 hair washes.

The use of color pigments is known for short-term color changes on hair. Pigments or color pigments are generally understood to mean insoluble dyeing substances. These are present undissolved in the form of small particles in the dye formulation and are deposited only from the outside onto the keratinous fibers and/or the hair surface. Therefore, they can generally be removed again without residue by a few washes using surfactant-containing cleaning agents. Various products of this type are available on the market under the name “hair mascara”.

If the user desires particularly long-lasting colors, the use of oxidative coloring agents has hitherto been the only option. However, despite multiple optimization attempts, an unpleasant ammonia odor or amine odor cannot be completely avoided in oxidative hair dyeing. The hair damage that remains associated with the use of the oxidative coloring agents also has a disadvantageous effect on the hair of the user. The search for alternative, high-performance dyeing methods is therefore an ongoing challenge. One possible alternative coloring system, which recently has been moving increasingly into focus, is based on the use of colored pigments.

Dyeing with pigments offers various major advantages. Since the pigments are deposited only from the outside to the keratinous fibers, in particular to the hair fibers, the damage associated with the dyeing process is very particularly low. Furthermore, desired colorings that are no longer desired can be removed quickly and easily without residue and therefore offer the user the possibility of returning directly and without great effort to their original hair color. This dyeing process is therefore particularly attractive for consumers who do not regularly want to re-dye their hair.

In current works, the problem of the low durability of this coloring system has been addressed. In this context, it was found that the wash fastness of the coloring results obtained with pigments could be greatly improved by combining the pigments with certain amino-functionalized silicone polymers. In addition, the choice of particularly well-suited pigments and pigment concentrations on dark hair achieved a brighter color result so that lightening was even possible with this coloring system, which was possible until now only with oxidative hair treatment agents (bleaching or decolorizing agents).

In addition to these many advantages, however, the coloring system based on pigments still has some disadvantages. Since both the pigments and the aminosilicones which immobilize the pigments are deposited on the surface of the hair fibers, their surface structure is modified by the formation of a film. Depending on the thickness of the formed film, this modification may also be associated with a change in the haptic impression of the hair fibers, ranging from a feeling of having heavier or greasy hair to a rough, shaggy or straw-like feel to the hair.

It was the object of the present invention to provide a coloring system based on pigments which allows intense color results with good hair feel. A technique has been sought that makes it possible to fix colored pigments to the hair as permanently as possible without the hair feeling heavy, greasy, unnatural, straw-like or coated. A particular focus of the object was to achieve intense, colorfast results with good hair feel at the same time.

Surprisingly, it has now been found that keratinous fibers, which had previously been dyed by application of aminosilicones and pigments, can be greatly improved in terms of their hair feel and their hair structure when they are post-treated with a specific post-treatment agent. The post-treatment agent is characterized in this case in that it contains at least one sebum-adsorbing substance.

The present invention relates firstly to a method for improving the tactile sensation of keratinous fibers which have been dyed by applying at least one amino-functionalized silicone polymer and at least one pigment, wherein a post-treatment agent is applied to the dyed keratinous fibers, which post-treatment agent contains:
(N-1) at least one sebum-adsorbing substance.

This is in particular a method for improving the tactile sensation of human hair which has been dyed by applying at least one amino-functionalized silicone polymer and at least one pigment, wherein a post-treatment agent is applied to the dyed hair and contains:
(N-1) at least one sebum-adsorbing substance.

The works leading to this invention have shown that keratin fibers, in particular hair, could be intensely dyed by applying pigments. Very particularly intense coloring results were obtained in this case when the coloring was carried out with a combination of pigment and aminosilicone. However, since the pigment or the mixture of pigment and aminosilicone deposits on the outside on the surface of the keratin fibers, the color result was associated with a deterioration of the haptic impression of the dyed fibers. The dyed hair feels coated and heavy, and the presence of the pigments resulted in a very rough, unnatural hair feel. If, moreover, larger amounts of aminosilicones were used in dyeing, they also left a greasy, heavy or oily impression on the hair. It has been unpredictably shown that the post-treatment with the post-treatment agent according to the invention leads to a significant improvement in the hair feel without the dyed hair suffering greater losses in terms of the color intensity.

Without being limited to this theory, it is assumed that the sebum-adsorbing substances contained in the post-treatment agent can adsorb or suck in not only the sebum formed naturally on the scalp but also the excess fractions of aminosilicones. In this way, the sebum-adsorbing substances are capable of reducing the greasy hair feel, but without disproportionately removing the film of aminosilicone and pigments necessary for the intense coloring. In other words, it has surprisingly been found that the sebum-adsorbing substances can absorb the excess fractions of aminosilicone without, however, impairing the intact nature of the film consisting of pigment and aminosilicone.

### Keratinous Fibers

Keratinous fibers are understood to mean hair, wool and fur. The keratinous fibers are preferably understood to mean hair, in particular human hair.

### Coloring Agent

In the context of this invention, the term “coloring agent” is used for a coloring of the keratin fibers, in particular of the hair, brought about by the use of pigments. In this coloring, the pigments are deposited, as dyeing compounds embedded in a film of aminosilicones, on the surface of the keratinous fibers. The terms keratinous fibers and keratin fibers are used synonymously in the context of the invention.

### Method for Improving the Tactile Sensation

An improvement of the tactile sensation is understood to mean that the dyed keratin fibers, in particular human hair, give a more natural and softer impression when the hair is touched, and that the dyed keratin fibers or the dyed hairs feel less heavy, greasy, oily, unnatural, straw-like, rough or coated.

The tactile sensation is a sensory impression which can be felt, for example, by trained persons such as hairdressers.

The improvement of the tactile sensation of a strand of hair can be determined, for example, by a hairdresser or also by an experienced user by feeling or touching two different strands and therefore determining by comparison which of the two strands feel better, i.e., which of the two strands makes a less greasy, oily, coated, rough or straw-like impression.

### Amino-Functionalized Silicone Polymer in the Coloring Agent

The post-treatment agent used in the method according to the invention showed a particularly strong effect when a combination of pigments with aminosilicones was used for coloring the keratin material or the keratin fibers.

The amino-functionalized silicone polymer can alternatively also be referred to as aminosilicone or amodimethicone.

Silicone polymers are generally macromolecules with a molecular weight of at least 500 g/mol, preferably at least 1000 g/mol, more preferably of at least 2500 g/mol, particularly preferably of at least 5000 g/mol, which comprise repeating organic units.

The maximum molecular weight of the silicone polymer depends on the degree of polymerization (number of polymerized monomers) and the batch size, and is also determined by the polymerization method. Within the meaning of the present invention, it is preferable if the maximum molecular weight of the silicone polymer is not more than 107 g/mol, preferably not more than 106 g/mol, and particularly preferably not more than 105 g/mol.

The silicone polymers comprise many Si-O repeat units, wherein the Si atoms can carry organic residues such as, for example, alkyl groups or substituted alkyl groups. Alternatively, a silicone polymer is therefore also referred to as polydimethylsiloxane.

In accordance with the high molecular weight of the silicone polymers, these are based on more than 10 Si—O repeat units, preferably more than 50 Si—O repeat units and particularly preferably more than 100 Si—O repeat units, very particularly preferably more than 500 Si—O units.

An amino-functionalized silicone polymer is understood to mean a functionalized silicone which carries at least one structural unit with an amino group. The amino-functionalized silicone polymer preferably carries a plurality of structural units with at least one amino group in each case. An amino group is understood to mean a primary amino group, a secondary amino group and a tertiary amino group. All of these amino groups can be protonated in an acidic environment and are then present in their cationic form.

In principle, a good coloring performance with amino-functionalized silicone polymers were achieved if these bear at least one primary, at least one secondary and/or at least one tertiary amino group. However, intense colorings with the best colorfastness were obtained when an amino-functionalized silicone polymer containing at least one secondary amino group had been used in the agent.

In a very particularly preferred embodiment, a method according to the invention is characterized in that the post-treatment agent is applied to keratinous fibers which were dyed by application of at least one amino-functionalized silicone polymer with at least one secondary amino group.

The secondary amino groups(s) may be at different positions of the amino-functionalized silicone polymer. Very particularly good color results were obtained when an amino-functionalized silicone polymer had been used that had at least one, preferably several structural units of the formula (Si-amino).

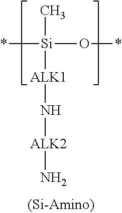

In the structural units of the formula (Si-amino), the abbreviations ALK1 and ALK2 are each independently a linear or branched divalent C₁-C₂₀ alkylene group.

In a further very particularly preferred embodiment, a method according to the invention is characterized in that the post-treatment agent is applied to keratinous fibers which have been dyed by application of at least one amino-functionalized silicone polymer which comprises at least one structural unit of formula (Si-amino),

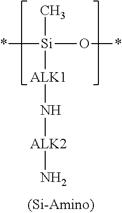

in which
ALK1 and ALK2 represent, independently of one another, a linear or branched divalent C₁-C₂₀ alkylene group.

The positions marked with an asterisk (*) in each case indicate the bond to further structural units of the silicone polymer. For example, the silicon atom adjacent to the asterisk can be bonded to a further oxygen atom, and the oxygen atom adjacent to the asterisk can be bonded to a further silicon atom or else to a C₁-C₆ alkyl group.

A divalent C₁-C₂₀ alkylene group can alternatively also be designated a divalent C₁-C₂₀ alkylene group, which means that each ALK1 or ALK2 grouping can form two bonds.

In the case of ALK1, the silicon atom is bonded to the grouping ALK1, and the second bond is between ALK1 and the secondary amino group.

In the case of ALK2, a bond from the secondary amino group to the grouping ALK2 takes place, and the second bond is between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. Above a chain length of 3 C atoms, divalent alkylene groups may also be branched. Examples of branched divalent C₃-C₂₀ alkylene groups are (—CH₂—CH(CH₃)—) and (—CH₂—CH(CH₃)—CH₂—).

In a further particularly preferred embodiment, the structural units of the formula (Si-amino) represent repeat units in the amino-functionalized silicone polymer, so that the silicone polymer comprises a plurality of structural units of the formula (Si-amino).

In the following, particularly well-suited amino-functionalized silicone polymers with at least one secondary amino group are listed.

Colorings with the best washing fastness could be obtained if at least one agent containing at least one amino-functionalized silicone polymer comprising structural units of formula (Si-I) and formula (Si-II) had been applied to the keratinous material during the preceding coloring.

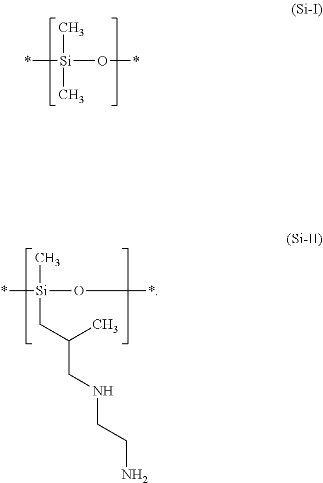

In a further explicitly very particularly preferred embodiment, a method according to the invention is characterized in that the post-treatment agent is applied to keratinous fibers which have been dyed by application of at least one amino-functionalized silicone polymer which comprises structural units of formula (Si-I) and formula (Si-II).

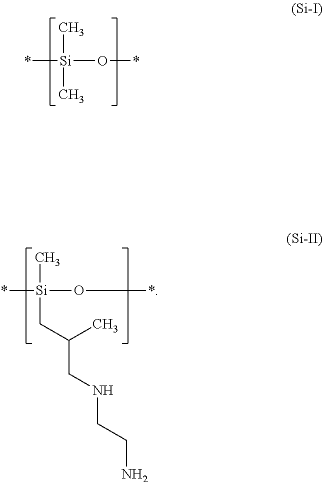

A corresponding amino-functionalized silicone polymer with the structural units (Si-I) and (Si-II) is, for example, the commercial product DC 2-8566 or Dowsil 2-8566 Amino Fluid, which is sold commercially by Dow Chemical Company and which carries the designation “Siloxanes and Silicones, 3-[(2-aminoethyl)amino]-2-methylpropyl Me, Di-Me-Siloxane” and the CAS number 106842-44-8. A further particularly preferred commercial product is Dowsil AP-8658 Amino Fluid, which is likewise commercially sold by Dow Chemical Company.

In the context of a further preferred embodiment, the post-treatment agent can also be applied to keratin fibers which were previously dyed by the application of a coloring agent containing at least one amino-functional silicone polymer of formula (Si-III),

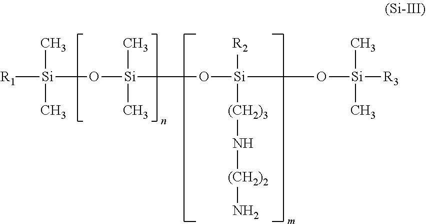

in which
m and n denote numbers that are selected such that the sum (n+m) is in the range of 1 to 1000,
n is a number in the range of 0 to 999 and m is a number in the range of 1 to 1000,
R1, R2 and R3, which are identical or different, denote a hydroxyl group or a C₁-4 alkoxy group,
at least one of the R1 to R3 groups denoting a hydroxyl group.

Further methods preferred according to the invention are characterized by the preceding application of a coloring agent to the keratin fibers, wherein the coloring agent contains at least amino-functional silicone polymer of formula (Si-IV),

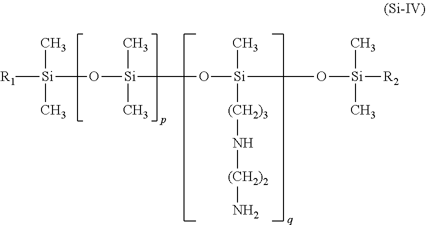

where
p and q denote numbers that are selected such that the sum (p+q) is in the range of 1 to 1000,
p is a number in the range of from 0 to 999 and q is a number in the range of 1 to 1000, and
R1 and R2, which are different, denote a hydroxy group or a C₁-4 alkoxy group, at least one of the groups R1 to R2 denoting a hydroxy group.

The silicones of formulas (Si-III) and (Si-IV) are different due to the grouping on the Si atom which carries the nitrogen-containing group: In formula (Si-III), R2 denotes a hydroxyl group or a C1-4 alkoxy group, while the residue in formula (Si-IV) is a methyl group. The individual Si groupings, which are labeled with the subscripts m and n or p and q, need not be present as blocks, but rather the individual units can also be distributed randomly; i.e., in the formulas (Si-III) and (Si-IV), each R1—Si(CH₃)₂ group is not necessarily bound to a —[O—Si(CH₃)₂] grouping.

Methods according to the invention in which a coloring agent containing at least one amino-functional silicone polymer of formula (Si-V) is applied to the keratin fibers have also proven to be particularly effective with regard to the generation of intense color results

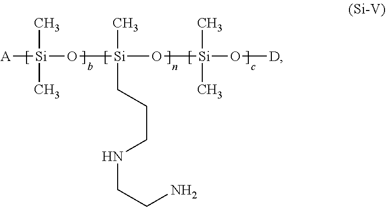

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c stand for integers between 0 and 1000,
with the proviso that
   n>0 and b+c>0
   at least one of the conditions A=—OH or D=—H is met.

In the above formula (Si-V), the individual siloxane units having the indices b, c and n are randomly distributed, i.e. they are not necessarily block copolymers.

The previously applied coloring agent can also contain one or more different amino-functionalized silicone polymers which are described by the formula (Si-VI):

M(RₐQ_{b}SiO_{(4-a-b)/2)x}(R_{c}SiO_{(4-c)/2)y}M (Si-VI)

wherein, in the above formula, R is a hydrocarbon or a hydrocarbon residue having 1 to approximately 6 carbon atoms, Q is a polar residue of general formula —R¹HZ in which R¹ is a bivalent, linking group that is bonded to hydrogen and the residue Z and composed of carbon and hydrogen atoms, carbon, hydrogen and oxygen atoms, or carbon, hydrogen and nitrogen atoms, and Z is an organic, aminofunctional residue containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably from approximately 3 to approximately 50, and most preferably from approximately 3 to approximately 25, and y is a number in a range of approximately 20 to approximately 10,000, preferably from approximately 125 to approximately 10,000, and most preferably from approximately 150 to approximately 1,000, and M is a suitable silicone end group as is known in the prior art, preferably trimethylsiloxy. Non-limiting examples of the residues represented by R include alkyl residues, such as methyl, ethyl, propyl, isopropyl, isopropyl, butyl, isobutyl, amyl, isoamyl, hexyl, isohexyl, and the like; alkenyl residues such as vinyl, halovinyl, alkylvinyl, allyl, haloallyl, and alkylallyl; cycloalkyl residues such as cyclobutyl, cyclopentyl, cyclohexyl, and the like; phenyl residues, benzyl residues, halohydrocarbon residues such as 3-chloropropyl, 4bromobutyl, 3,3,3-trifluoropropyl, chlorocyclohexyl, bromophenyl, chlorophenyl, and the like, and sulfur-containing residues such as mercaptoethyl, mercaptopropyl, mercaptohexyl, mercaptophenyl, and the like; R is preferably an alkyl residue, containing 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —CH₂)₃C(O)SCH₂CH₂—.

Z is an organic, amino-functional residue containing at least one functional amino group. A possible formula for Z is NH(CH₂)_{z}NH₂, where z is 1 or more. Another possible formula for Z is —NH(CH₂)_{z}(CH₂)_{zz}NH, where both z and zz are independently 1 or more, this structure comprising diamino ring structures such as piperazinyl. Z is most preferably a —NHCH₂CH₂NH₂ residue. Another possible formula for Z is N(CH₂)_{z}(CH₂)_{zz}NX₂ or —NX₂, wherein each X of X₂ is selected independently from the group consisting of hydrogen and alkyl groups having 1 to 12 carbon atoms, and zz is 0.

Q is most preferably a polar, amino-functional residue of the formula —CH₂CH₂CH₂NHCH₂CH₂NH₂. In the formulas, “a” assumes values in the range of about 0 to about 2, “b” assumes values in the range of about 2 to about 3, “a”+ “b” is less than or equal to 3, and “c” is a number in the range of about 1 to about 3. The molar ratio of the RₐQ_{b} SiO_{(4-a-b)/2} units to the R_{C}SiO_{(4-c)/2} units is in the range of about 1:2 to 1:65, preferably from about 1:5 to about 1:65 and most preferably from about 1:15 to about 1:20. If one or more silicones of the above formula are used, then the various variable substituents in the above formula can be different in the various silicone components, present in the silicone mixture.

In the context of a particularly preferred embodiment, a method according to the invention is characterized by the preceding application of a coloring agent to the keratinous fibers, wherein the coloring agent is an amino-functional silicone polymer of formula (Si-VII):

R′ₐG₃₋ₐ-Si(OSiG₂)ₙ-(OSiG_{b}R′_{2-b})ₘ—O—SiG₃₋ₐ-R′ₐ (Si-VII),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃,
a represents a number between 0 and 3, in particular 0;
b represents a number between 0 and 1, in particular 1;
m and n are numbers whose sum (m+n) is between 1 and 2000, preferably between 50 and 150, n preferably assuming values of from 0 to 1999 and in particular from 49 to 149 and m preferably assuming values of from 1 to 2000, in particular from 1 to 10;
R′ is a monovalent residue selected from
   Q-N(R″)—CH₂—CH₂—N(R″)₂
   Q-N(R″)₂
   Q-N⁺(R″)₃A⁻
   Q-N⁺H(R″)₂A⁻
   Q-N⁺H₂(R″)A⁻
   Q-N(R″)—CH₂—CH₂—N⁺R″H₂A_{″}
      where each Q represents a chemical bond, —CH₂—, —CH₂—CH—₂—, —CH₂CH₂CH₂—, —C(CH₃)₂—, —CH₂CH₂CH₂CH₂—, —CH₂C(CH₃)₂—, or —CH(CH₃)CH₂CH₂—, R″ represents identical or different residues from the group —H, -phenyl, -benzyl, —CH₂—CH(CH₃)Ph, C₁-20-alkyl residue, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂ CH₃, —C(CH₃)₃, and A represents an anion preferably selected from chloride, bromide, iodide or methosulfate.

In the context of a further preferred embodiment, a method according to the invention is characterized by the preceding application of a coloring agent to the keratinous fibers, wherein the coloring agent contains at least one amino-functional silicone polymer of formula (Si-VIIa),

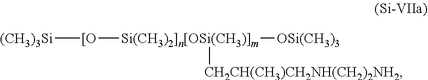

in which m and n are numbers whose sum (m+n) is between 1 and 2000, preferably between 50 and 150, n preferably assuming values of from 0 to 1999 and in particular from 49 to 149 and m preferably assuming values of from 1 to 2000, in particular from 1 to 10.

These silicones are designated as trimethylsilylamodimethicones in accordance with the INCI declaration.

In the context of a further preferred embodiment, a method according to the invention is characterized by the preceding application of a coloring agent to the keratinous fibers, wherein the coloring agent contains at least one amino-functional silicone polymer of formula (Si-VIIb),

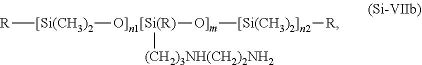

in which R represents —OH, —O—CH₃, or a —CH₃ group and m, n1 and n2 are numbers whose sum (m+n1+n2) is between 1 and 2000, preferably between 50 and 150, the sum (n1+n2) preferably assuming values of from 0 to 1999 and in particular from 49 to 149 and m preferably assuming values of from 1 to 2000, in particular from 1 to 10.

These amino-functionalized silicone polymers are designated as amodimethicones in accordance with the INCI declaration.

Irrespective of which amino-functional silicones are used, coloring agents according to the invention that contain an amino-functional silicone whose amine value is above 0.25 meq/g, preferably above 0.3 meq/g, and in particular above 0.4 meq/g are preferred. The amine value here represents the milliequivalents of amine per gram of the amino-functional silicone. Said value can be determined by titration and may also be given in the unit mg KOH/g.

Furthermore, coloring agents which contained a specific 4-morpholinomethyl-substituted silicone polymer are also suitable for use in the method according to the invention. This amino-functionalized silicone polymer comprises structural units of the formulas (SI-VIII) and of the formula (Si-IX).

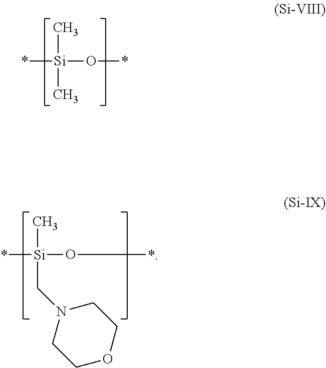

Corresponding 4-morpholinomethyl-substituted silicone polymers are described below.

A corresponding amino-functionalized silicone polymer is known under the name Amodimethicone/Morpholinomethyl Silsesquioxane Copolymer and is commercially available in the form of the raw material Belsil ADM 8301 E from Wacker.

For example, a silicone which has structural units of the formulas (Si-VIII), (Si-IX) and (Si-X) can be used as 4-morpholinomethyl-substituted silicone

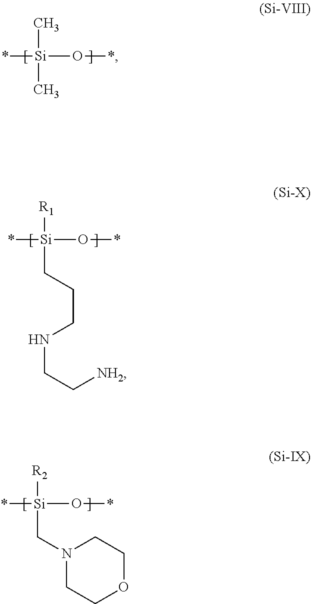

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred coloring agents contain at least one 4-morpholinomethyl-substituted silicone of formula (Si-XI)

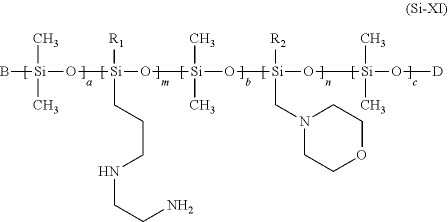

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃;
B represents an —OH, —O—Si(CH₃)₃, —O—Si(CH₃)₂OH, or —O—Si(CH₃)₂OCH₃ group;
D represents an —H, —Si(CH₃)₃, —Si(CH₃)₂OH, —Si(CH₃)₂OCH₃ group;
a, b and c represent, independently of one another, integers between 0 and 1000, with the proviso that a+b+c>0;
m and n represent, independently of one another, integers between 1 and 1000, with the proviso that
   at least one of the conditions B=—OH or D=—H is met,
   the units a, b, c, m and n are distributed statistically or in blocks in the molecule.

Structural formula (Si-XI) is intended to indicate that the siloxane groups n and m do not necessarily have to be directly bound to an end group B or D. Instead, in preferred formulas (Si-VI), a>0 or b>0 and, in particularly preferred formulas (Si-VI), a>0 and c>0; i.e. the terminal group B or D is preferably bound to a dimethylsiloxy group. In formula (Si-VI) as well, the siloxane units a, b, c, m and n are preferably randomly distributed.

The silicones represented by formula (Si-VI) and used according to the invention can be trimethylsilyl-terminated (D or B=—Si(CH₃)₃), but they may also be dimethylsilylhydroxy-terminated at both ends or dimethylsilylhydroxy-terminated at one end and dimethylsilylmethoxy-terminated at the other end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃and D=—Si(CH₃)₂OH
B=—O—Si(CH₃)₂OCH₃ and D=—Si(CH₃)₂OH.

These silicones lead to enormous improvements in the hair properties of hair treated with the agents according to the invention, and to greatly improved protection during oxidative treatment.

The agent used in the method according to the invention for prior dyeing may contain one or more amino-functionalized silicone polymers for example in a total amount of from 0.1 to 8.0 wt. %, preferably from 0.2 to 5.0 wt. %, more preferably from 0.3 to 3.0 wt. % and very particularly preferably from 0.4 to 2.5 wt. %. Here, the indicated amounts relate to the total amount of all aminosilicones used, which is set in relation to the total weight of the coloring agent.

In this context, it was observed that the impression of greasy, heavy or oily hair also depends on the amount of aminosilicones used. The higher the content of aminosilicone in the coloring agent, the more negative was the haptic impression. The use of the post-treatment agent according to the invention showed a particularly strong improvement in the hair feel if a very high weight fraction of aminosilicones was used in the prior application of the coloring agent.

In the context of another particularly preferred embodiment, a method according to the present invention is characterized in that the coloring agent—based on the total weight of the coloring agent—contains one or more amino-functionalized silicone polymers in a total amount of 0.1 to 8.0 wt %, preferably 0.4 to 5.0 wt %, more preferably 0.8 to 3.0 wt %, and very particularly preferably 1.0 to 3.5 wt %.

### Pigments in the Coloring Agent

In the method according to the invention, a post-treatment agent is applied to keratin fibers which were previously dyed by application of at least one pigment.

Pigments within the meaning of the present invention are understood to mean dyeing compounds which have a solubility in water, at 25° C., of less than 0.5 g/L, preferably of less than 0.1 g/L, even more preferably of less than 0.05 g/L. The water solubility can be carried out, for example, by means of the method described hereinafter: 0.5 g of the pigment is weighed out in a beaker. A stirring bar is added. Then, one liter of distilled water is added. This mixture is heated to 25°° C., while stirring on a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be present finely dispersed, the mixture is filtered. If a proportion of undissolved pigments remains on the filter paper, the solubility of the pigment is below 0.5 g/L.

Suitable dye pigments may be of inorganic and/or organic origin. In a preferred embodiment, a method according to the invention is characterized in that the post-treatment agent is applied to keratin material which has been dyed by application of at least one inorganic and/or organic pigment.

Preferred dye pigments are selected from synthetic or natural inorganic pigments. Inorganic dye pigments of natural origin can be produced, for example, from chalk, ocher, umbra, green earth, burnt sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred dye pigments are black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI77289), iron blue (ferric ferrocyanide, CI77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to prepare the pearlescent pigments in conjunction with metal oxides, the mica, primarily muscovite or phlogopite, is coated with a metal oxide.

As an alternative to natural mica, synthetic mica coated with one or more metal oxides(s) can optionally also be used as the pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In a preferred embodiment, a method according to the invention is characterized in that the post-treatment agent is applied to keratinous fibers which have been dyed by application of at least one inorganic pigment, the inorganic pigment preferably being selected from the group of colored metal oxides, metal hydroxides, metal oxide hydrates, silicates, metal sulfides, complex metal cyanides, metal sulphates, bronze pigments and/or from mica-based colored pigments which are coated with at least one metal oxide and/or metal oxychloride.

In a preferred embodiment, a method according to the invention is characterized in that the post-treatment agent is applied to keratin fibers which have been dyed by application of at least one pigment selected from mica-based pigments which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable dye pigments are commercially available for example under the trade names Rona®, Colorona®, Xirona®, Dichrona® and Tiimron® from the company Merck, Ariabel® and Unipure® from the company Sensient, Prestige® from the company Eckart, Cosmetic Colors and Sunshine® from the company Sunstar.

Very particularly preferred dye pigments having the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (Iron oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide) Colorona Bright Gold, Merck, Mica, CI 77891 (Titanium dioxide), CI 77491 (Iron oxides) Colorona Blackstar Gold, Merck, MICA, CI 77499 (IRON OXIDES)

Further particularly preferred dye pigments having the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magie Mauve, Merck, Silica, CI 77891 (Titanium Dioxide), Tin Oxide.

In addition, particularly preferred dye pigments having the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In the context of a further embodiment, the previously applied coloring agent can also contain one or more organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

For example carmine, quinacridone, phthalocyanine, sorghum, blue pigments having the Color Index Numbers CI 42090, CI 69800, CI 69825, CI 73000, CI 74100, CI 74160, yellow pigments having the Color Index Numbers CI 11680, CI 11710, CI 15985, CI 19140, CI 20040, CI 21100, CI 21108, CI 47000, CI 47005, green pigments having the Color Index Numbers CI 61565, CI 61570, CI 74260, orange pigments having the Color Index Numbers CI 11725, CI 15510, CI 45370, CI 71105, red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470 can be cited as particularly well-suited organic pigments.

In a further particularly preferred embodiment, a method according to the invention is characterized in that the post-treatment agent is applied to keratinous fibers which have been dyed by application of at least one organic pigment, the organic pigment preferably being selected from the group of carmine, quinacridone, phthalocyanine, sorghum, blue pigments having the Color Index Numbers CI 42090, CI 69800, CI 69825, CI 73000, CI 74100, CI 74160, yellow pigments having the Color Index Numbers CI 11680, CI 11710, CI 15985, CI 19140, CI 20040, CI 21100, CI 21108, CI 47000, CI 47005, green pigments having the Color Index Numbers CI 61565, CI 61570, CI 74260, orange pigments having the Color Index Numbers CI 11725, CI 15510, CI 45370, CI 71105, red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can furthermore also be a color varnish. The term color varnish is understood, within the meaning of the invention, to mean particles which comprise a layer of adsorbed dyes, the unit consisting of particle and dye being insoluble under the above-mentioned conditions. The particles may be, for example, inorganic substrates, which may be aluminum, silica, calcium borosilicate, calcium aluminum borosilicate or aluminum.

For example, the Alizarin color varnish can be used as color varnish.

For the dyeing of the keratin fibers, pigments of a specific shape may also have been used. For example, a pigment based on a lamellar and/or lenticular substrate platelet may be used. Furthermore, the dyeing is also possible based on a small substrate plate which comprises a vacuum metalized pigment.

In the context of a further embodiment, a method according to the invention is characterized in that the post-treatment agent is applied to keratinous fibers which were dyed by application of at least one pigment from the group of pigments based on a lamellar substrate platelet, pigments based on a lenticular substrate platelet, and vacuum-metalized pigments.

The small substrate plates of this type have an average thickness of at most 50 nm, preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each small substrate plate has as uniform a thickness as possible.

Due to the small thickness of the small substrate plates, the pigment has a particularly high covering capacity.

The substrate platelets are preferably constructed monolithically. Monolithic means, in this context, consisting of a single closed unit without fractures, layers or inclusions, it being possible, however, for structural changes to occur within the small substrate plates. The small substrate plates are preferably constructed homogeneously, i.e. no concentration gradient occurs within the small plates. In particular, the small substrate plates are not constructed in layers and have no particles distributed therein.

The size of the small substrate plate can be matched to the respective application, in particular to the desired effect on the keratin material. In general, the substrate platelets have an average largest diameter of approximately 2 to 200 pm, in particular approximately 5 to 100 pm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Heios type, with Quixel wet dispersion. In this case, for sample preparation, the sample to be investigated was pre-dispersed in isopropanol for a period of 3 minutes.

The small substrate plates may be constructed from any material that can be made into the form of a small plate.

They can be of natural origin, but can also be produced synthetically. Materials from which the small substrate plates can be constructed are, for example, metals and metal alloys, metal oxides, preferably aluminum oxide, inorganic compounds, and minerals such as mica and (semi-)precious stones, as well as plastics materials. Preferably, the small substrate plate are made of metal (alloy)s.

Any metal suitable for metallic pearlescent pigments is possible as the metal. Such metals are, inter alia, iron and steel, and all air-resistant and water-resistant (semi-)metals such as, for example, platinum, zinc, chromium, molybdenum and silicon, as well as alloys thereof such as aluminum bronzes and brass. Preferred metals are aluminum, copper, silver and gold. Preferred small substrate plates are small aluminum plates and small brass plates, small substrate plates made of aluminum being particularly preferred.

Lamellar small substrate plates are characterized by an irregularly structured edge, and are also referred to as “cornflakes” due to their appearance.

Due to their irregular structure, pigments based on lamellar small substrate plates produce a high fraction of scattered light. In addition, the pigments based on lamellar small substrate plates do not completely cover the existing color of a keratin material and, for example, effects can be achieved analogously to a natural graying.

Small lenticular (=lens-shaped) substrate plates have a substantially regular round edge and are also referred to as “silver dollars” due to their appearance. Due to their regular structure, the fraction of the reflected light predominates in pigments based on small lenticular substrate plates.

Vacuum metalized pigments (VMP) can be obtained, for example, by releasing metals, metal alloys or metal oxides from correspondingly coated films. These are characterized by a particularly small thickness of the small substrate plates in the range from 5 to 50 nm and by a particularly smooth surface having increased reflectivity. Small substrate plates which comprise a pigment metalized in vacuo are also referred to, within the context of this application, as VMP small substrate plates. VMP small substrate plates made of aluminum can be obtained, for example, by releasing aluminum from metalized films.

The small substrate plates made of metal or metal alloy can be passivated, for example by anodizing (oxide layer) or chromatizing.

Uncoated lamellar, lenticular and/or VPM substrate platelets, in particular those made of metal or metal alloy, reflect the incident light to a high degree and produce a light-dark flop. These have proven to be particularly preferred for use in the coloring agent.

Suitable pigments based on a lamellar substrate platelet include, for example, the pigments of the VISIONAIRE series by Eckart.

Pigments based on a lenticular substrate platelet are available, for example, under the name Alegrace® Gorgeous from the company Schlenk Metallic Pigments GmbH.

Pigments based on a substrate platelet, which comprises a vacuum metalized pigment, are available, for example, under the name Alegrace® Marvelous or Alegrace® Aurous from the company Schlenk Metallic Pigments GmbH.

Due to their excellent light and temperature resistance, the use of the aforementioned pigments in the agent according to the invention is very particularly preferred. It is furthermore preferred if the pigments used have a specific particle size. It is therefore advantageous according to the invention if the at least one pigment has an average particle size D₅₀ from 1.0 to 50 μm, preferably from 5.0 to 45 μm, preferably from 10 to 40 μm, in particular from 14 to 30 μm. The average particle size D₅₀ can be determined, for example, using dynamic light scattering (DLS).

The agent used in the method according to the invention for prior dyeing may contain one or more pigments, for example in a total amount of from 0.01 to 10.0 wt. %, preferably from 0.1 to 5.0 wt. %, more preferably from 0.2 to 2.5 wt. % and very particularly preferably from 0.25 to 1.5 wt. %. Here, the indicated amounts relate to the total amount of all pigments used, which is set in relation to the total weight of the coloring agent.

In another very particularly preferred embodiment, a method according to the invention is characterized in that the post-treatment agent is applied to keratinous fibers which have been dyed by application of at least one coloring agent containing—based on the total weight of the coloring agent—one or more pigments in a total amount of 0.01 to 10.0 wt %, preferably 0.1 to 5.0 wt %, more preferably 0.2 to 2.5 wt %, and very particularly preferably 0.25 to 1.5 wt %.

As a further optional component, the coloring agents may additionally also contain one or more direct dyes. The direct dyes are dyes which attach directly to the hair and require no oxidative process to form the color. Direct dyes are usually nitrophenylenediamines, nitroaminophenols, azo dyes, anthraquinones, triarylmethane dyes, or indophenols.

The direct dyes within the meaning of the present invention have a solubility in water (760 mmHg) at 25°° C. of more than 0.5 g/L and are therefore not to be regarded as pigments. Within the meaning of the present invention, the direct dyes preferably have a solubility in water (760 mmHg) at 25° C. of more than 1.0 g/L.

Direct dyes can be divided into anionic, cationic, and non-ionic direct dyes.

In a further embodiment, a coloring agent according to the invention can be characterized in that it additionally contains at least one dyeing compound from the group of anionic, non-ionic and cationic direct dyes.

Suitable cationic direct dyes are Basic Blue 7, Basic Blue 26, HC Blue 16, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

In particular, non-ionic nitro dyes and quinone dyes and neutral azo dyes, for example, can be used as non-ionic direct dyes. Suitable non-ionic direct dyes are the compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and also 1,4-diamino-2-nitrobenzene, 2-amino-4-nitrophenol, 1,4-bis (2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

Anionic direct dyes are also referred to as acid dyes. Acid dyes are understood to mean direct dyes having at least one carboxylic acid group (—COOH) and/or one sulfonic acid group (—SO3H). Depending on the pH, the protonated forms (—COOH, —SO₃H) of carboxylic acid or sulfonic acid groupings are present in equilibrium with their deprotonated forms (—COO—, —SO₃). The proportion of the protonated forms increases with a decreasing pH. If direct dyes are used in the form of their salts, therefore the carboxylic acid groups or sulfonic acid groups are present in the deprotonated form and are neutralized to maintain the electroneutrality with corresponding stoichiometric equivalents of cations. Acid dyes according to the invention can also be used in the form of their sodium salts and/or their potassium salts.

The acid dyes within the meaning of the present invention have a solubility in water (760 mmHg) at 25°° C. of more than 0.5 g/L and are therefore not to be regarded as pigments. Within the meaning of the present invention, the acid dyes preferably have a solubility in water (760 mmHg) at 25° C. of more than 1.0 g/L.

The alkaline earth salts (for example calcium salts and magnesium salts) or aluminum salts of acid dyes often have poorer solubility than the corresponding alkali salts. If the solubility of these salts is below 0.5 g/L (25° C., 760 mmHg), they do not fall under the definition of a direct dye.

An essential feature of the acid dyes is their ability to form anionic charges, the carboxylic acid groups or sulfonic acid groups responsible for this usually being linked to different chromophoric systems. Suitable chromophoric systems are found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In the context of a further embodiment, an agent for dyeing keratinous material can be characterized in that it contains at least one anionic direct dye which is selected from the group of nitrophenylenediamines, the nitroaminophenols, the azo dyes, the anthraquinone dyes, the triarylmethane dyes, the xanthene dyes, the rhodamine dyes, the oxazine dyes and/or the indophenol dyes, wherein the dyes from the aforementioned group each contain at least one carboxylic acid group (—COOH), a sodium carboxylate group (—COONa), a potassium carboxylate group (—COOK), a sulfonic acid group (—SO₃H) a sodium sulfonate group (—SO₃Na) and/or a potassium sulfonate group (—SO₃K).

For example, one or more compounds can be selected as suitable acid dyes from the following group: Acid Yellow 1 (D&C Yellow 7, Citronin A, Ext. D&C Yellow No. 7, Japan Yellow 403, CI 10316, COLIPA no. B001), Acid Yellow 3 (COLIPA no.: C 54, D&C Yellow no. 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no. C₀₁₅), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No.201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Echtrot D, FD&C Red No. 2, Food Red 9, Naphtholrot S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, lodeosin), Acid Red 52 (CI 45100, Food Red 106, Solar Rhodamine B, Acid Rhodamine B, Red no. 106 Pontacyl Brilliant Pink), Acid Red 73 (CI CI 27290), Acid Red 87 (Eosin, CI 45380), Acid Red 92 (COLIPA no. C₅₃, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no. 2, C.I. 60730, COLIPA no C₀₆₃), Acid Violet 49 (CI 42640), Acid Violet 50 (CI 50325), Acid Blue 1 (Patent Blue, CI 42045), Acid Blue 3 (Patent Blau V, CI 42051), Acid Blue 7 (CI 42080), Acid Blue 104 (CI 42735), Acid Blue 9 (E 133, Patentblau AE, Amidoblau AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, FoodgreenI), Acid Green 5 (CI 42095), Acid Green 9 (C.1.42100), Acid Green 22 (C.1.42170), Acid Green 25 (CI 61570, Japan Green 201, D&C Green No. 5), Acid Green 50 (acid brilliant green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The water solubility of the anionic direct dyes can be determined, for example, in the following manner. 0.1 g of the anionic direct dye is placed in a beaker. A stirring bar is added. Then 100 ml of water are added. This mixture is heated to 25° C. on a magnetic stirrer, while stirring. The mixture is stirred for 60 minutes. Thereafter, the aqueous mixture is visually assessed. If there are still undissolved residues, the amount of water is increased, for example in steps of 10 ml. Water is added until the amount of dye used has dissolved completely. If the dye-water mixture cannot be visually assessed due to the high intensity of the dye, the mixture is filtered. If a proportion of undissolved dyes remains on the filter paper, the solubility test is repeated with a larger amount of water. If 0.1 g of the anionic direct dye dissolves in 100 ml of water at 25° C., then the solubility of the dye is 1.0 g/L.

Acid Yellow 1 carries the name 8-hydroxy-5,7-dinitro-2-naphthalenesulfonic acid disodium salt and has a solubility in water of at least 40 g/L (25° C.).

Acid Yellow 3 is a mixture of the sodium salts of mono-and di-sulfonic acids of 2-(2-quinolyl)-1H-indene-1,3(2H)-dione and has a water solubility of 20 g/L (25° C.).

Acid Yellow 9 is the disodium salt of 8-hydroxy-5,7-dinitro-2-naphthalenesulfonic acid, its water solubility is above 40 g/L (25° C.).

Acid Yellow 23 is the trisodium salt of 4,5-dihydro-5-oxo-1-(4-sulfophenyl)-4-((4-sulfophenyl)azo)-1H-pyrazole-3-carboxylic acid, and is readily soluble in water at 25° C.

Acid Orange 7 is the sodium salt of 4-[(2-hydroxy-1-naphthyl)azo]benzene sulphonate. Its water solubility is more than 7 g/L (25° C.).

Acid Red 18 is the trisodium salt of 7-hydroxy-8-[(E)-(4-sulfonato-1-naphthyl)diazenyl)]-1,3-naphthalenedisulfonate and has a very high water solubility of more than 20 wt. %.

Acid Red 33 is the disodium salt of 5-amino-4-hydroxy-3-(phenylazo)-naphthalene-2,7-disulfonate, its water solubility is 2.5 g/L (25°° C.).

Acid Red 92 is the disodium salt of 3,4,5,6-tetrachloro-2-(1,4,5,8-tetrabromo-6-hydroxy-3-oxanthene-9-yl) benzoic acid, the water solubility of which is specified as greater than 10 g/L (25° C.).

Acid Blue 9 is the disodium salt of 2-({4-[N-ethyl(3-sulfonatobenzyl]amino]phenyl}{4-(N-ethyl(3-sulfonatobenzyl)imino]-2,5-cyclohexadien-1-ylidene}methyl)-benzenesulfonate and has a water solubility of more than 20 wt. % (25° C.).

In a further embodiment, a method according to the invention is therefore characterized in that the post-treatment agent is applied to keratinous fibers which have been dyed by application of at least one coloring agent which contains at least one direct dye selected from the group consisting of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct dye(s) can be used in different amounts in the coloring agent depending on the desired color intensity. Good results were able to be obtained if the coloring agent contains—based on the total weight of the coloring agent—one or more direct dyes in a total amount of from 0.01 to 10.0 wt. %, preferably from 0.1 to 8.0 wt. %, more preferably from 0.2 to 6.0 wt. %, and very particularly preferably from 0.5 to 4.5 wt. %.

Furthermore, the agent can also contain a dyeing compound from the group of photochromic or thermochromic dyes as an additional optional component.

Photochromic dyes are dyes which react to irradiation with UV light (sunlight or black light) with a reversible color tone change. The UV light changes the chemical structure of the dyes and therefore their absorption behavior (photochromia).

Thermochromic dyes are dyes which react to temperature changes with a reversible color tone change. The temperature change changes the chemical structure of the dyes and therefore their absorption behavior (thermochromia).

The coloring agent contains—based on the total weight of the agent—one or more photochromic and/or thermochromic coloring agents in a total amount of 0.01 to 10.0 wt. %, preferably 0.1 to 8.0 wt. %, more preferably 0.2 to 6.0 wt. %, and very particularly preferably 0.5 to 4.5 wt. %.

### Post-Treatment Agent

In the context of the method according to the invention, a post-treatment agent is applied to the keratin fibers, in particular to human hair, which were dyed as described above. This application of the post-treatment agent is associated with a significant improvement in the feel of the hair. The time at which the post-treatment agent is applied depends on the needs of the user and can be adapted to their habits.

For example, it is possible for post-treatment agents to be applied to the freshly dyed, still wet or just dried keratin fibers, so that a period of only a few minutes to a few hours is between the rinsing out of the coloring agent and the application of the post-treatment agent. Furthermore, the user can also decide to color the hair the day before and apply the post-treatment agent only on the next day. In this case, the post-treatment agent can be applied to the wet or dry hair dyed on the day before.

Furthermore, it is possible for a longer period of time to be present between the prior application of the coloring agent and the application of the post-treatment agent, which can be a few to several days or even weeks. In this context, the requirement applies that the post-treatment agent is applied to dyed keratin material, which means that the keratin material must still be dyed by the application of the pigments.

It is very particularly preferred if the coloring and the post-treatment are carried out within the scope of a single coloring post-treatment process so that a period of a maximum of a few hours, preferably of a maximum of a few hours, preferably of a maximum of 2 hours, is present between the coloring of the keratin fibers and the time at which the post-treatment agent is applied.

### Sebum-Adsorbing Substance in the Post-Treatment Agent

Characteristic for the post-treatment agent according to the invention is its content of at least one sebum-adsorbing substance (N-1).

The sebum-adsorbing substance can alternatively also be referred to as a tallow-adsorbing substance. A sebum-adsorbing substance is a compound which is capable of adsorbing, i.e. receiving, the sebum or tallow produced on the human scalp. In this case, the sebum accumulates on the surface or inside this substance. In the prior art, sebum-adsorbing substances are often described as active ingredients in dry shampoos.

The sebum-adsorbing substances can be used in different forms in the post-treatment agent according to the invention. The use as a sebum or tallow adsorbing powder has proven to be particularly advantageous.

Whether a powder is sebum-adsorbing can be determined, for example, via the tallow absorbing capacity of this powder. The sebum/tallow absorption corresponds here to the amount of sebum adsorbed by the powder. It is expressed in ml tallow per 100 g of powder, measured according to a method for determining the oil absorption of powders, as described, for example, in various standards known from the prior art.

The oil absorption of the powder corresponds to the amount of sebum adsorbed on the available surface of the powder, which can be determined, for example, by measuring the “wet point” according to the following measurement method.

An amount m (in grams) of powder is applied to a glass plate, wherein the amount depends on the density of the powder. Then, artificial tallow is added dropwise, composed of 29 wt. % of triolein, 28.5 wt. % of oleic acid, 18.5 wt. % of oleyl oleate, 14 wt. % of squalene, 7 wt. % of cholesterol and 3 wt. % of cholesterol palmitate. After addition of 4 to 5 drops of the artificial tallow, it is worked into the powder with a spatula, and the addition of artificial tallow is continued until a conglomerate forms from artificial tallow and powder. From this time, the artificial tallow is added dropwise, and the mixture is subsequently mixed with the spatula. The addition of artificial tallow is stopped when a solid, smooth paste has formed. This paste must be distributed without cracks on the glass plate, i.e. the paste must spread on the glass plate without cracks or lumps forming. The volume of the artificial tallow in ml required for this process is noted and converted into the corresponding tallow absorption of the unit ml/100 g.

Particularly effective sebum-adsorbing powders are capable of a sebum absorption of at least 35 ml/100 g.

When the sebum-adsorbing substances are used in the form of a powder, it has proven expedient to reduce the water content of the post-treatment agent to a maximum of 10 wt. %.

In a preferred embodiment, the post-treatment agent used in the method according to the invention is characterized in that it has a water content of less than 10 wt. %, preferably less than 5 wt. %, even more preferably less than 1 wt. %, based on the total weight of the post-treatment agent.

In the context of a further very particularly preferred embodiment, a method according to the invention is characterized in that the post-treatment agent has a water content of less than 10 wt. %, preferably less than 5 wt. %, based on the total weight of the post-treatment agent, and (N-1) contains at least one sebum-adsorbing powder.

A water content of less than 10 wt. % in the post-treatment agent means that the amount of water in the post-treatment agent which comes about by the direct use of water in the agent and/or which is introduced into the post-treatment agent by the amounts of water present in the ingredients used is below 10 wt. %.

The sebum or tallow-absorbing powder can be, for example, a mineral or an organic powder. A powder is a mixture of small particles solid at 25° C. and normal pressure (1013 mbar).

The sebum-adsorbing substance or the sebum-adsorbing powder can be selected from:
starches, such as starch powders,
modified starches, such as starch octenyl succinates and in particular aluminum starch octenyl succinates, or starch phosphates such as distarch phosphate,
talc
silicic acids and silica,
calcium silicates,
perlites,
zeolites,
polylactic acids,
polyamide powders (Nylon®),
acrylic polymer powders, in particular polymethyl methacrylate, poly(methyl methacrylate/ethylene glycol dimethacrylate), poly(allyl methacrylate/ethylene glycol dimethacrylate), ethylene glycol dimethacrylate/lauryl methacrylate copolymer,
elastomeric silicone powder,
   and mixtures thereof.

In the context of a very particularly preferred embodiment, a method according to the invention is characterized in that the post-treatment agent contains:

(N-1) at least one sebum-adsorbing substance from the group of starches, modified starches, talc, silicic acids, silica, calcium silicates, perlites, zeolites, polylactic acids, polyamide powders and acrylic polymer powders.

The sebum-adsorbing powder may also be a powder coated with a hydrophobic treatment agent. The hydrophobic treatment agent can be selected from the group consisting of fatty acids such as stearic acid, metal soaps such as aluminum dimyristate, the aluminum salt of hydrogenated tallow glutamate, amino acids such as aluminum dimyristate, aluminum salt of hydrogenated tallow glutamates; amino acids; n-acylated amino acids or their salts; lecithin, isopropyltrisostearyl titanate and mixtures thereof.

N-acylated amino acids can contain an acyl group having 8 to 22 carbon atoms, such as 2-ethylhexanoyl, caproyl, lauroyl, myristoyl, palmitoyl, stearoyl, and cocoyl.

The salts of these compounds may be aluminum, magnesium, calcium, zirconium, zinc, sodium or potassium salts. The amino acid may be, for example, lysine, glutamic acid or alanine.

Starch is a reserve carbohydrate which is stored by many plants in the form of starch grain (granules) usually 1 to 200 μm in size in different plant parts, for example in tubers or roots, cereal seeds, fruits and in the pith. Starch belongs to the family of homoglycans and is a polycondensation product of D-glucose. Starch consists of three structurally different polymers of d-glucopyranose, namely amylose, amylopectin and a so-called intermediate fraction.

A starch compound which is preferably usable according to the invention is selected from at least one—optionally modified—polycondensation product of D-glucose obtained from starch from rice, corn, potatoes, peas, acorns, chestnuts, barley, wheat, bananas, sago, millet, sorghum, oats, rye, beans, batata, arrowroot or cassava.

The post-treatment agent according to the invention particularly preferably contains at least one starch compound (N-1) from the group consisting of rice starch, corn starch, potato starch, and tapioca starch. Mixtures of the aforementioned starch compounds are also included according to the invention. The starch compound is very particularly preferably rice starch. Starch compounds based on rice starch are, for example, obtainable under the name Remy DR KA (INCI name: Oryza Sativa (Rice) Starch, CAS number 9005-25-8) from Bene O Remy Industries or under the name Rice Starch D.S.A. 7 (INCI name: Oryza Sativa (Rice) Starch, Cetrimonium Chloride, CAS number 9005-25-8) from Agrana.

In the context of a very particularly preferred embodiment, a method according to the invention is characterized in that the post-treatment agent contains:
(N-1) at least one sebum-adsorbing substance from the group consisting of starches from rice, corn, potatoes, peas, acorns, chestnuts, barley, wheat, banana, sago, millet, sorghum, oats, rye, beans, batata, arrowroot or cassava, very particularly preferably from the group of rice starch, corn starch, potato starch, tapioca starch, wheat starch, and cassava starch.

In the context of a very particularly preferred embodiment, a method according to the invention is characterized in that the post-treatment agent has a water content of less than 10 wt. %, preferably less than 5 wt. %, based on the total weight of the post-treatment agent, and: (N-1) at least one sebum-adsorbing substance from the group consisting of starches from rice, corn, potatoes, peas, acorns, chestnuts, barley, wheat, banana, sago, millet, sorghum, oats, rye, beans, batata, arrowroot or cassava, very particularly preferably from the group of rice starch, corn starch, potato starch, tapioca starch, wheat starch, and cassava starch.

A modified starch can also be used as the starch. A modified starch is a starch modified by a method known to a person skilled in the art, such as esterification, etherification, oxidation, acidic hydrolysis, crosslinking or enzymatic conversion.

Non-limiting examples of modified starches are aluminum starch octenyl succinate, sodium starch octenyl succinate, sodium starch octenyl succinate, calcium starch octenyl succinate, starch phosphate, distarch phosphate, hydroxyethyl starch phosphate, hydroxypropyl starch phosphate, sodium carboxymethyl starch, and sodium glycolate starch.

In one particular embodiment, the modified starch is a starch octenyl succinate, in particular aluminum starch octenyl succinate. Aluminum starch octenyl succinate is a starch modified with octenylsuccinic anhydride, which is present in the form of its aluminum salt. Aluminum starch octenyl succinate bears the CAS No. 9087-61-0 and is commercially available, for example, under the trade name Covafluid AMD from Sensient. The product which is commercially sold by AKZO NOBEL under the name DRY FLO PLUS is also particularly noteworthy.

Starch phosphates are derivatives of starch belonging to the starch esters. Monostarch phosphate bears the designation E 1410 and distarch phosphate the designation E 1412. Starch phosphate is prepared by reacting starch with monosodium orthophosphate or disodium orthophosphate in a polymer-analogous reaction.

In the case of starch monophosphate, the bond is preferably formed at the Ce atom of the glucose building block. This compound is produced, for example, by treatment of starch with monosodium orthophosphate or disodium orthophosphate or alkali tripolyphosphates at pH 5.5-6 and 120-175°° C. over 1-15 h.

In the case of distarch phosphate, the phosphate group forms a bridge between two starch chains. The preparation is carried out by reacting native starch in aqueous suspension with sodium trimetaphosphate, phosphoroxychloride or phosphorus pentachloride in the presence of sodium chloride, sodium carbonate or sodium hydroxide at pH 10-11. After the reaction has ended, a pH of between 4.5-6 is set, and the product is then washed with water and dried or converted to swelling starch. In the case of distarch phosphate, the degree of substitution is lower by approximately one power of ten than with starch monophosphate. Distarch phosphate has the INCI name Distarch Phosphate and bears the CAS number 55963-33-2.

Distarch phosphate can be purchased commercially, for example, under the trade name PB 2000 from Nippon Starch Chemical.

In the context of another particularly preferred embodiment, a method according to the invention is characterized in that post-treatment agent contains: (N-1) at least one sebum-adsorbing substance from the group of modified starches, preferably from the group of aluminum starch octenylsuccinate and starch phosphates.

Another sebum-adsorbing substance according to the invention is talc or talcum, preferably talc with the INCI name Talc, E553b. Talc is a natural, widely used magnesium silicate Mg₃[(OH)₂/Si₄O_{10]} or 3MgO 4SiO₂H₂O, which belongs to the three-layer (2:1) phyllosilicates, the denser aggregates of which are called soapstone.

Further sebum-adsorbing substances according to the invention can be selected from the group of silicic acids. In this case, both hydrophilic silicic acids and hydrophobic silicic acids can be used.

It is particularly preferred to use mixtures of at least one hydrophilic silicic acid and at least one hydrophobic silicic acid. It is particularly preferred here if the hydrophobic silicic acid(s) are present in excess in relation to the hydrophilic silicic acid(s). Preference is given to hydrophobic: hydrophilic weight ratios from 0:1 to 10:1, preferably 2:1 to 6:1, particularly preferably 3:1 to 4:1.

Hydrophilic silicic acids used according to the invention are easily wettable with water. Preferred hydrophilic silicic acids are hydrophilic fumed silicic acids, in particular the non-alkylated commercial products of the Aerosil® series by Evonik Degussa, in particular Aerosil® 130, Aerosil® 150, Aerosil® 200, Aerosil® 300, Aerosil® 380, also products of the Cab—O—Sil series from Cabot, in particular Cab—O—Sil HS-5.

A suitable hydrogenated silica is also the product commercially available under the trade name Sipernat 22S by Evonik. This raw material is described as amorphous silica and bears the CAS number 7631-86-9 or 112926-00-8.

Hydrophobic silicic acids used according to the invention are alkyl-modified at least on the surface of the silicic acid particles. They preferably contain hydrophobic groups on the surface, such as (CH₃)₃Si—O—, and (—Si(CH₃)₂O—)ₙ.

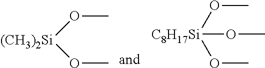

Preferred hydrophobic silicic acids are hydrophobic fumed silicic acids, in particular the alkylated commercial products of the Aerosil® series by Evonik Degussa, in particular Aerosil® R202, Aerosil® R805, Aerosil® R812, Aerosil® R972 and Aerosil® R976, also products of the Cab—O—Sil TS series by Cabot, in particular Cab—O—Sil TS-530.

Particularly preferred hydrophobic silicic acids are silica silylate and silica dimethyl silylate. Particularly preferred is, furthermore, Aerosil® R972, which has a BET surface area of approximately 110 m²/g and has about 70% of the surface occupied with methylated hydroxyl groups.

Another sebum-adsorbing substance according to the invention is perlite. Perlite refers to a vulcanized glass (obsidian).

Further sebum-adsorbing substances according to the invention can be selected from the group of zeolites. Zeolites are crystalline aluminosilicates of the general formula M_{2/z}O□Al₂O₃□xSiO₂□yH₂O, with M=mono- or polyvalent metal ion (usually an alkali or alkaline earth cation), and also H or NH₄, z=valence of the cation, x=1.8 to about 12 and y=0 to about 8.

Important properties of the zeolites are their framework and pore structure and the acid centers and mobile ions incorporated into the framework. The crystal lattices of the zeolites are constructed of SiO₄ and AlO₄ tetrahedra, which are each linked to one another via oxygen bridges and form rings or prisms. These in turn combine to form further secondary building units (SBU), which can each contain up to 16 Si or Al atoms, from which a large structural diversity follows. This results in a spatial arrangement of identically constructed cavities which are accessible via windows (pore openings) or three-dimensional channel systems.

The stoichiometric ratio of Si to Al (modulus) is an important parameter of the zeolites. Since only SiOAl or SiOSi bridges, but never AIOAI bridges in the zeolite lattice, are permitted according to the so-called Löwenstein's rule, the modulus never falls below 1. With each exchange of an Si⁴⁺ by an Al³⁺ ion, a localized negative charge is introduced into the crystal lattice.

The zeolite minerals arise by hydrothermal conversion from vulcanized glasses or from tuffaceous deposits. According to their crystal lattices, the natural zeolites can be divided into:
fibrous zeolites (natrolite, lamontite, mordenite, thomsonite),
sheet zeolites (heulandite, stilbite, phillipsite, harmotome, yugawaralite)
and the so-called cube zeolites (faujasite, gmelinite, chabazite, offretite, levynite), which have a cubic or pseudo-cubic lattice.

The synthetic zeolites are divided according to pore widths into narrow-, medium- and wide-pored types.

Further sebum-adsorbing substances according to the invention can be selected from the group of polylactic acids. Polylactic acid, also referred to as polylactide or PLA, is one name for biologically degradable polymers (polyesters) that are accessible above all by the ionic polymerization of lactide, an annular combination of two lactic acid molecules.

At temperatures between 140 and 180° C. and also under the effect of catalytic tin compounds (for example tin oxide), a ring-opening polymerization occurs. Plastics having a high molecular mass and strength are therefore produced. Lactide itself can be produced by fermentation of molasses or by fermentation of glucose with the aid of various bacteria. In addition, high-molecular and pure polylactides can be produced directly from lactic acid with the aid of what is known as polycondensation. In industrial production, however, the disposal of the solvent is problematic.

The lactic acid (2-hydroxypropanoic acid) has an asymmetrical C atom, and therefore the polylactic acid also has optically active centers in L(+) and D(−) configuration. The ratio of L to D monomer units here determines the degree of crystallinity, the melting point, and the biological degradability of the polymers.

Polylactic acids that are suitable in accordance with the invention are L-polylactic acid, D-polylactic acid, and L/D polylactic acid, and mixtures thereof. The polylactic acid is preferably used in the form of particles or in the form of a powder in the post-treatment agent according to the invention.

The molar mass of the polylactic acid suitable in accordance with the invention is preferably 1,000 to 1,000,000, preferentially 10,000 to 300,000, more preferably 50,000 to 250,000, and in particular 100,000 to 180,000 Dalton.

In a further preferred embodiment of the present invention, the polylactic acid is used in a form blended with fillers. The use of larger filler quantities helps to comminute the polymer into particles, and increases the biodegradability and the internal specific surface area via porosity and capillarity. In particular, water-soluble fillers are preferred here, for example metal chlorides such as NaCl, KCl, etc., metal carbonates such as Na₂CO₃, NaHCO₃, etc., and metal sulfates such as MgSO₄.

As fillers, natural raw materials can also be used, for example nut shells, wood or bamboo fibers, starch, xanthan, alginates, dextran, agar, etc. These fillers are biodegradable and do not impair the good ecological properties of the polylactic acid particles.

Polylactic acid particles that are suitable in accordance with the invention can be present both in spherical form and in the form of irregular particles which have a certain circularity.

Further sebum-adsorbing substances according to the invention can be selected from the group of polyamides. Polyamides are linear polymers with regularly repeating amide bonds along the main chain. The amide group can be regarded as a condensation product of a carboxylic acid and an amide. The resulting bond is an amide bond which can be cleaved again hydrolytically.

A suitable polyamide is, for example, nylon 6.6 polyamide with the CAS number 32131-17-2, which is commercially available under the trade name of Nylon Polyamide Fibre 0.1 inch from Celllusude Products (Pacific Coast Chemical).

### Formulation of the Post-Treatment Agent

As already described, the post-treatment agent containing the sebum-adsorbing substances (N-1) is used to improve the tactile sensation of the keratinous fibers dyed in the previously described manner.

In this case, the post-treatment agent can be prepared in various ways and used by the user, wherein the type of preparation determines both the proportion of the sebum-adsorbing substances (N-1) in the post-treatment agent and the content of further optional ingredients.

The preparation of the post-treatment agent as a spray, in particular an aerosol spray, has proven to be very particularly suitable, since the sebum-adsorbing substances (N-1) can be sprayed in this way particularly uniformly onto the dyed keratin fibers.

When the post-treatment agent is prepared in the form of a spray or an aerosol spray, one or more adsorbing substances (N-1) are preferably suspended in a solvent, such as ethanol, and sprayed onto the keratin fibers by means of a blowing agent.

In the context of a further particularly preferred embodiment, a method according to the invention is characterized in that a post-treatment agent is sprayed onto the dyed keratin fibers and contains, based on the total weight of the post-treatment agent:
(N-1) 2.0 to 20 wt. % sebum-absorbing substance(s), preferably selected from the group of starch and modified starches,
(N-2) 2.0 to 20 wt. % ethanol, and
(N-3) 60 to 96 wt. % blowing agent.

Suitable blowing agents can be selected, for example, from the group of propane, propane/butane mixtures and dimethyl ether, in particular from the group of propane/butane mixtures.

Further suitable blowing agents or propellant gases are nitrogen, air, oxygen, nitrous oxide, 1,1,1,3-tetrafluoroethane, heptafluoro-n-propane, perfluoroethane, monochloro-difluoromethane, 1,1-difluoroethane, both individually and in combination. Hydrophilic blowing agents or propellant gases, such as carbon dioxide, can be used advantageously in accordance with the present invention if the proportion of hydrophilic gases is selected to be low, and lipophilic propellant gas (e.g., propane/butane) is present in excess.

In the context of a further particularly preferred embodiment, a method according to the invention is characterized in that a post-treatment agent is sprayed onto the dyed keratin fibers and contains, based on the total weight of the post-treatment agent:
(N-1) 3.0 to 8 wt. % sebum-absorbing substance(s), preferably selected from the group of starch and modified starches,
(N-2) 2.0 to 12 wt. % ethanol, and
(N-3) 80 to 96 wt. % blowing agent.

The ingredients (N-1), (N-2) and (N-3) add up here to a maximum of 100 wt. %, wherein the added weight sum thereof in the presence of further optional constituents can also decrease to values below 100 wt. %.

Furthermore, very good results were obtained if the post-treatment agent itself was also provided in the form of a powder and then scattered by the user as a powder onto the keratin fibers or hair, or was distributed on the hair with the aid of a powder pump. The dosage form of the post-treatment agent in the form of a powder additionally only provides very low demands on the packaging, so that it can be designed in a particularly material-saving and lasting manner.

When the post-treatment agent is prepared as a powder, it has been found to be advantageous to use the sebum-absorbing substance(s) (N-1) in higher weight fractions, which are preferably within a range from 50.0 to 100.0 wt. %, preferably from 60.0 to 99.9 wt. %, more preferably from 70.0 to 99.8 wt. % and very particularly preferably from 80.0 to 99.7 wt. %.

In the context of a further particularly preferred embodiment, a method according to the invention is characterized in that a powdered post-treatment agent is applied to the dyed keratin fibers and contains, based on the total weight of the post-treatment agent:
(N-1) one or more sebum-absorbing substances in a total amount of from 50.0 to 100.0 wt. %, preferably from 60.0 to 99.9 wt. %, more preferably from 70.0 to 99.8 wt. %, and very particularly preferably from 80.0 to 99.7 wt. %.

In the context of a further particularly preferred embodiment, a method according to the invention is characterized in that a powdered post-treatment agent is applied to the dyed keratin fibers and contains:
(N-11) one or more sebum-absorbing substances from the group of starches and modified starches, and
(N-12) one or more sebum-absorbing substances from the group of silicic acids and silica.

In the context of a further particularly preferred embodiment, a method according to the invention is characterized in that a powdered post-treatment agent is applied to the dyed keratin fibers and contains, based on the total weight of the post-treatment agent:
(N-11) one or more sebum-absorbing substances from the group of starches and modified starches in a total amount of 60 to 95 wt. %, preferably 70 to 90 wt. %, and
(N-12) one or more sebum-absorbing substances from the group of silicic acids and silica in a total amount of 5 to 40 wt. %, preferably from 10 to 30 wt. %.

### Further Optional Ingredients in the Post-Treatment Agent

In addition to the above-described components essential to the invention, the post-treatment agent according to the invention can optionally also contain one or more further ingredients.

Further optional ingredients are, for example, cationic, non-ionic, zwitterionic and/or anionic surfactants, further solvents, anionic, non-ionic, zwitterionic and/or cationic polymers; structurants such as glucose, maleic acid and lactic acid, hair-conditioning compounds such as phospholipids, for example lecithin and cephalins; perfume oils, dimethyl isosorbide and cyclodextrins; fiber structure-improving agents, in particular mono-, di- and oligosaccharides, for example glucose, galactose, fructose and lactose; dyes for coloring the product; anti-dandruff active ingredients such as piroctone olamine, zinc omadine and climbazole; amino acids and oligopeptides; animal and/or vegetable-based protein hydrolysates, as well as in the form of their fatty acid condensation products or optionally anionically or cationically modified derivatives; plant oils, light stabilizers and UV blockers; active ingredients such as panthenol, pantothenic acid, pantolactone, allantoin, pyrrolidinone carboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; perfumes, plant extracts; fats and waxes such as fatty alcohols, beeswax, montan wax and paraffins; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono-and distearate as well as PEG-3-distearate.

The selection of these additional substances is made by a person skilled in the art according to the desired properties of the agents. With regard to other optional components and the amounts of said components used, reference is explicitly made to relevant reference books known to a person skilled in the art. The additional active ingredients and auxiliaries are used in the preparations according the invention preferably in each case in amounts from 0.0001 to 25 wt. %, in particular from 0.0005 to 15 wt. %, based on the total weight of the particular agent.

### Method for Dyeing and Post-Treating Keratinous Material, in Particular Human Hair

As already described above, the time at which the post-treatment agent is applied to the dyed hair can be freely selected depending on the preferences of the user. It can be particularly convenient for the user to carry out the coloring of the hair and the application of the post-treatment agent in directly successive steps within an application process.

The present invention therefore relates secondly to a method for dyeing keratinous fibers, in particular human hair, comprising the following steps:
(1) applying a coloring agent to the keratinous fibers, the coloring agent containing at least one amino-functionalized silicone polymer and at least one pigment as already described in detail with the description of the first subject matter of the invention,
(2) exposing the coloring agent applied in step (1) to the keratinous fibers,
(3) rinsing out the coloring agent with water,
(4) optionally drying the keratinous fibers,
(5) applying a post-treatment agent to the dyed keratinous fibers, wherein the post-treatment agent has already been disclosed in detail in the description of the first subject of the invention.

In step (1) of the method according to the invention, a coloring agent containing at least one amino-functionalized silicone polymer and at least one pigment, in particular the previously described preferred and particularly preferred representatives thereof, is applied to the keratinous fibers, in particular hair.

In the subsequent step (2), the previously applied coloring agent is left to act on the hair. However, a great advantage of the coloring system according to the invention is that an intense color result can be achieved even in very short periods after short exposure times. For this reason, it is advantageous for the coloring agent to remain on the keratin material after its application only for comparatively short periods from 30 seconds to 15 minutes, preferably from 30 seconds to 10 minutes, and particularly preferably from 1 to 5 minutes.

In a further preferred embodiment, a method according to the invention is characterized by the:
(2) exposure of the coloring agent applied in step (1) to the keratinous fibers for a period from 30 seconds to 15 minutes, preferably from 30 seconds to 10 minutes, and particularly preferably from 1 to 5 minutes.

Following the exposure of the coloring agent on the keratin material, it is lastly rinsed with water in step (3). In a preferred embodiment, the coloring agent is washed out only with water, i.e. without the aid of a post-treatment agent or a shampoo not according to the invention.

Optionally, the dyed keratinous fibers can then be dried in step (4). Drying can take place, for example, by using a heating hood or a hairdryer. If the keratin fibers are dried, step (5) takes place after step (4). If the keratin fibers are not dried, step (5) takes place after step (3).

Subsequently, the post-treatment agent is applied, in particular in its previously described preferred and particularly preferred embodiments, in step (5).

In this context, it has proved preferable to apply post-treatment agents to the hair within a period of a maximum of 5 hours, particularly preferably within a period of a maximum of 2 hours after the coloring agent has been washed out.

In a further preferred embodiment, a method according to the invention is characterized by the:
(5) application of a post-treatment agent to the dyed keratinous fibers within a period of not more than 3 hours after washing out of the coloring agent in step (3).

The post-treatment agent is particularly preferably applied to the dried or dry hair after step (4).

The exposure of the post-treatment agent to the keratinous fibers in step (5) can take place for example for a period from 15 seconds to 10 minutes, preferably for a period from 30 seconds to 5 minutes.

Optionally, after step (5), the post-treatment agent can be combed out of the keratinous fibers in step (6). By combing out, the sebum-absorbing substances (N-1) are removed from the keratin fibers after the excess fractions of aminosilicone have been absorbed. The combing out can be done, for example, with the aid of a comb or a brush.

In a further preferred embodiment, a method according to the invention is characterized in that it comprises, as a further step:
(6) combing out the post-treatment agent applied in step (5).

A preferred method for dyeing keratinous fibers, in particular human hair, comprises the following steps in the indicated sequence:
(1) applying a coloring agent to the keratinous fibers, the coloring agent containing at least one amino-functionalized silicone polymer and at least one pigment as already described in detail with the description of the first subject matter of the invention,
(2) exposing the coloring agent applied in step (1) to the keratinous fibers,
(3) rinsing out the coloring agent with water,
(4) drying the keratinous fibers,
(5) applying a post-treatment agent to the dyed keratinous fibers, wherein the post-treatment agent has already been disclosed in detail for the description of the first subject matter of the invention, and
(6) combing out the post-treatment agent applied in step (5).

### Multi-Component Packaging Unit

To increase the user comfort, the user is preferably provided with all the required agents in the form of a multicomponent packaging unit (kit-of-parts).

A further subject of the present invention is therefore a multi-component packaging unit (kit-of-parts) for dyeing and post-treating keratinous fibers, in particular human hair, comprising, packaged separately from one another:
a first container with a coloring agent, the coloring agent containing at least one amino-functionalized silicone polymer and at least one pigment as already disclosed in detail in the description of the first subject of the invention, and
a second container with a post-treatment agent, wherein the post-treatment agent has already been disclosed in detail in the description of the first subject matter of the invention.

A third subject of the present invention is therefore a multi-component packaging unit (kit-of-parts) for dyeing and post-treating keratinous fibers, in particular human hair, comprising, packaged separately from one another:
a first container with a first agent which contains at least one amino-functionalized silicone polymer as already disclosed in detail in the description of the first subject of the invention, and
a second container with a second agent, which contains at least one pigment as already disclosed in detail in the description of the first subject of the invention, and
a third container with a post-treatment agent, as has already been disclosed in detail in the description of the first subject of the invention.

Concerning the additional preferred embodiments of the multi-component packaging unit according to the invention, what has been said about the method according to the invention applies mutatis mutandis.

### Use

A further subject of the present invention is the use of a post-treatment agent, as has already been disclosed in detail in the description of the first subject matter of the invention, for improving the tactile sensation of dyed keratinous fibers, in particular dyed human hair.

Concerning the additional preferred embodiments of the use according to the invention, what has been said about the method according to the invention applies mutatis mutandis.

### EXAMPLES

1. Coloring agent
The following coloring agents were produced (all
information is in wt. % unless indicated otherwise)

| Coloring agent | F1 | F2 | F3 | F4 |
|---|---|---|---|---|
| Alegrace Marvelous D 12/77-1 Shiny Silver (Pigment) | 4.8 | 2.5 | 2.5 | 2.5 |
| Unipure Red LC 381 (Pigment) | — | 0.5 | — | — |
| Goldmann Pigment Blue 60 (Pigment) | — | — | 0.2 | — |
| Unipure Red LC 3079 (Pigment) | — | — | — | 0.4 |
| Plantacare 2000 UP | — | 0.5 | — | — |
| Water, demineralized | — | 3.9 | — | — |
| UCON FLUID AP (PPG-14 Butyl Ether) | — | 1.0 | 1.0 | 1.0 |
| PPG-3 Benzyl Ether Myristate | — | — | — | 3.31 |
| DOWSIL AP-8568 Amino Fluid (amino-functionalized | 0.74 | 0.74 | 0.74 | 0.74 |
| silicone polymer) | | | | |
| Polyethylene Glycol 6000 | 9.5 | 19.4 | 19.4 | 19.4 |
| Polyethylene Glycol MG 400 | up to 100 | up to 100 | up to 100 | up to 100 |
| Coloring agent | F5 | F6 | F7 | F8 |
| Alegrace Marvelous D 12/77-1 Shiny Silver (Pigment) | — | — | | 1.2 |
| Unipure Red LC 381 (Pigment) | 0.48 | — | — | 0.5 |
| Goldmann Pigment Blue 60 (Pigment) | — | 0.18 | — | 0.1 |
| Unipure Red LC 3079 (Pigment) | — | — | 0.37 | 0.5 |
| Plantacare 2000 UP | 0.5 | — | — | 0.6 |
| Water, demineralized | 3.9 | — | — | 3.9 |
| UCON FLUID AP (PPG-14 Butyl Ether) | 1.0 | 1.0 | 1.0 | 1.0 |
| PPG-3 Benzyl Ether Myristate | — | — | 3.31 | — |
| DOWSIL AP-8568 Amino Fluid (amino-functionalized | 0.74 | 0.74 | 0.74 | 0.74 |
| silicone polymer) | | | | |
| Polyethylene Glycol 6000 | 9.5 | 19.4 | 19.4 | 19.4 |
| Polyethylene Glycol MG 400 | up to 100 | up to 100 | up to 100 | up to 100 |

2. Post-treatment agent
The following coloring agents were produced (all
information is in wt. % unless indicated otherwise)

| Post-treatment agent in spray form | N1 |
|---|---|
| Ethanol | 10.0 |
| Perfume | 0.1 |
| Rice starch | 5.0 |
| Cetrimonium chloride | 0.005 |
| Blowing agent (propane/butane 15:85) | up to 100 |
| Post-treatment agent in powder form | N2 |
| Perfume | 0.4 |
| Silicic acid, pyrogenic | 15.0 |
| Tapioca starch | 82.6 |
| Sipernat 22 S (hydrogenated silica, | up to 100 |
| amorphous silica, Evonik) | |

### 2. Application

The coloring agent was applied in each case to strands of hair and left to act for three minutes. Subsequently, the strands of hair were washed thoroughly (1 minute) with water and then dried with a hairdryer.

As soon as the strands of hair were dry, the post-treatment agent was applied to the dyed strands of hair. The post-treatment agent in spray form was added to a pressure vessel and sprayed onto the strands of hair. The post-treatment agent in powder form was scattered onto the dyed strands of hair and then manually worked into the strands of hair.

After 5 minutes, each strand of hair was combed out. The tactile sensation of the dry hair was assessed by trained individuals.

| Coloring agent | Post-treatment agent |
|---|---|
| Coloring agent F1 | N1 (spray) |
| Coloring agent F1 | N2 (powder) |
| Coloring agent F2 | N1 (spray) |
| Coloring agent F2 | N2 (powder) |
| Coloring agent F3 | N1 (spray) |
| Coloring agent F3 | N2 (powder) |
| Coloring agent F4 | N1 (spray) |
| Coloring agent F4 | N2 (powder) |
| Coloring agent F5 | N1 (spray) |
| Coloring agent F5 | N2 (powder) |
| Coloring agent F6 | N1 (spray) |
| Coloring agent F6 | N2 (powder) |
| Coloring agent F7 | N1 (spray) |
| Coloring agent F7 | N2 (powder) |
| Coloring agent F8 | N1 (spray) |
| Coloring agent F8 | N2 (powder) |

By post-treatment with the post-treatment agents N1 and N2, the hair feel of all hair strands that had been dyed with the coloring agents F1 to F8 could be significantly improved.

1. A method for improving the tactile sensation of keratinous fibers, the method comprising:
applying a post-treatment agent to keratinous fibers which have been dyed with at least one amino-functionalized silicone polymer and at least one pigment,
wherein the post-treatment agent comprises at least one sebum-adsorbing substance.

2. The method of claim 1, wherein the post-treatment agent is applied to the keratinous fibers which have been dyed with the at least one amino-functionalized silicone polymer having at least one secondary amino group.

3. The method of claim 1, wherein the post-treatment agent is applied to the keratinous fibers which have been dyed with the at least one amino-functionalized silicone polymer having at least one structural unit of formula (Si-amino): 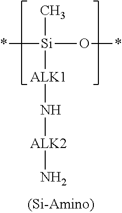
where
ALK1 and ALK2 represent, independently of one another, a linear or branched divalent C₁-C₂₀ alkylene group.

4. The method of claim 1, wherein the post-treatment agent is applied to the keratinous fibers which have been dyed with the at least one amino-functionalized silicone polymer having structural units of formula (Si-I) and of formula (Si-II).

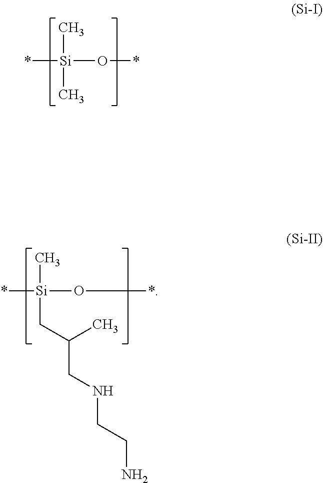

5. The method of claim 1, wherein the at least one pigment is at least one inorganic pigment is selected from the group consisting of colored metal oxides, metal hydroxides, metal oxide hydrates, silicates, metal sulfides, complex metal cyanides, metal sulphates, bronze pigments, and mica-based colored pigments coated with at least one metal oxide and/or metal oxychloride.

6. The method of claim 1, wherein the at least one pigment is at least one organic pigment, selected from the group consisting of carmine, quinacridone, phthalocyanine, sorghum, blue pigments having the Color Index Numbers Cl 42090, CI 69800, CI 69825, CI 73000, CI 74100, or CI 74160, yellow pigments having the Color Index Numbers CI 11680, CI 11710, CI 15985, CI 19140, CI 20040, CI 21100, CI 21108, CI 47000, or CI 47005, green pigments having the Color Index Numbers CI 61565, CI 61570, or CI 74260, orange pigments having the Color Index Numbers CI 11725, CI 15510, CI 45370, or CI 71105, and red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, or CI 75470.

7. The method of claim 1, wherein the at least one pigment is selected from the group consisting of pigments based on a lamellar substrate platelet, pigments based on a lenticular substrate platelet, and vacuum-metalized pigments.

8. The method of claim 1, wherein the post-treatment agent less than 10 wt. % of water based on the total weight of the post-treatment agent.

9. The method of claim 1, wherein the at least one sebum-adsorbing substance is selected from the group consisting of starches, modified starches, talc, silicic acids, silica, calcium silicates, perlites, zeolites, polylactic acids, polyamide powders, and acrylic polymer powders.

10. The method of claim 1, wherein the at least one sebum-adsorbing substance is selected from the group consisting of starches from rice, corn, potatoes, peas, acorns, chestnuts, barley, wheat, banana, sago, millet, sorghum, oats, rye, beans, batata, arrowroot, and cassava.

11. The method of claim 1, wherein at least one sebum-adsorbing substance is selected from the group consisting of aluminum starch octenylsuccinate and starch phosphates.

12. The method of claim 1, wherein the post-treatment agent comprises:
2.0 to 20 wt. % of the at least one sebum-absorbing substance based on the total weight of the post-treatment agent,
2.0 to 20 wt. % of ethanol based on the total weight of the post-treatment agent, and
60 to 96 wt. % of a blowing agent based on the total weight of the post-treatment agent.

13. The method of claim 1, wherein the post-treatment agent is powdered and comprises 50.0 to 100.0 wt. % of the at least one sebum-adsorbing substance based on the total weight of the post-treatment agent.

14. A method for dyeing and post-treating keratinous fibers, the method comprising:
applying a coloring agent to the keratinous fibers, the coloring agent comprising at least one amino-functionalized silicone polymer and at least one pigment;
exposing the coloring agent to the keratinous fibers;
rinsing the coloring agent out of the keratinous fibers;
drying the keratinous fibers dyed with the coloring agent; and
applying a post-treatment agent to the dyed keratinous fibers, the post-treatment agent comprising at least one sebum-adsorbing substance.

15. The method of claim 14, further comprising combing the post-treatment agent out of the dyed keratinous fibers.

16. A multi-component packaging for dyeing and post-treating keratinous fibers, the multi-component packaging comprising:
a first container containing a first agent comprising at least one amino-functionalized silicone polymer;
a second container containing a second agent comprising at least one pigment; and
a third container containing a post-treatment agent comprising at least one sebum-adsorbing substance,
wherein the first container, the second container, and the third container are packaged separately from one another.

17. (canceled)

18. The method of claim 1, further comprising applying the at least one amino-functionalized silicone polymer and the at least one pigment to the keratinous fibers before the application of the post-treatment agent.

19. The method of claim 1, wherein the keratinous fibers are human hair.

20. The method of claim 1, wherein the at least one sebum-adsorbing substance is at least one sebum-adsorbing powder.

21. The method of claim 14, wherein the keratinous fibers are human hair.
